(19) United States
(12) Patent Application Publication
(10) Pub. No.: US 2014/0066759 A1
(43) Pub. Date: Mar. 6, 2014

(54) SYSTEMS AND METHODS FOR PARAMETRIC IMAGING

(71) Applicant: Andrew John Healey, Moss (NO)

(72) Inventor: Andrew John Healey, Moss (NO)

(73) Assignee: GENERAL ELECTRIC COMPANY, Schenectady, NY (US)

(21) Appl. No.: 13/602,759

(22) Filed: Sep. 4, 2012

(51) Int. Cl. A61B 8/13; A61B 8/08

(52) U.S. Cl. 600/431

(57) ABSTRACT

A method is provided including obtaining ultrasound information including anatomy information representative of a portion of anatomy to be imaged and artifact information representative of a coherent interference artifact. A contrast agent has been associated with the coherent interference artifact. Due to association with the contrast agent, the coherent interference artifact appears in different realizations in a plurality of readings taking along a single line. The method also includes suppressing the artifact information to form revised ultrasound information. Suppressing the artifact information includes using the plurality of readings to suppress the artifact information. Also, the method includes reconstructing the ultrasound image using the revised ultrasound information.

### BACKGROUND OF THE INVENTION

The subject matter disclosed herein relates generally to image reconstruction, and more particularly to systems and methods for using ultrasound contrast agents to produce a parametric image related to tissue attenuation properties.

Images of a subject, for example regions of interest of a patient, may be obtained by a variety of different methods. Such methods include, as examples, ultrasound, single photon emission computed tomography (SPECT), positron emission tomography (PET), magnetic resonance imaging (MRI), and computed tomography (CT). These imaging systems typically form an image by performing one or more data acquisitions at discrete time intervals, with an image formed from a combination of the information obtained by the data acquisitions. Ultrasound data acquisition systems are generally less expensive, more portable, and more readily available than such systems as SPECT, PET, MRI, and CT. Further, ultrasound data acquisition does not require any exposure to ionizing radiation as may be required by certain other scanning technologies.

However, in comparison to certain other imaging techniques, conventional ultrasound techniques, for example, may provide lower resolution images and/or may not provide adequate detection of certain parameters or characteristics of tissue being imaged. Additionally, noise or artifacts experienced by such ultrasound imaging techniques may prevent the identification of certain types of tissue or differences between tissue.

### BRIEF DESCRIPTION OF THE INVENTION

In accordance with various embodiments, a method for reconstructing an ultrasound image (and/or characterizing tissue) is provided. The method includes obtaining ultrasound information including anatomy information representative of a portion of anatomy to be imaged and artifact information representative of a coherent artifact source that has been associated with a contrast agent. Obtaining the ultrasound information includes taking a plurality of readings along a single line. Due to association with the contrast agent, the coherent interference artifact appears in different realizations in the plurality of readings. The method also includes suppressing the artifact information to form revised ultrasound information. Suppressing the artifact information includes using the plurality of readings along the single line to suppress the artifact information. Also, the method includes reconstructing the ultrasound image using the revised ultrasound information, and/or providing a parameter that characterizes the tissue type.

In accordance with other embodiments, a tangible and non-transitory computer readable medium comprising one or more computer software modules is provided. The one or more computer software modules are configured to direct a processor to receive ultrasound information from an ultrasound detector. The ultrasound information includes anatomy information representative of a portion of anatomy to be imaged and artifact information representative of a coherent interference artifact. The coherent interference artifact has been associated with a contrast agent. The ultrasound information comprises information obtained from a plurality of reading along a single line. Due to association with the contrast agent, the coherent interference artifact appears in different realizations in the plurality of readings. The one or more computer software modules are also configured to direct the processor to suppress the artifact information to form revised ultrasound information by using the plurality of readings along the single line. The one or more computer software modules are further configured to direct the processor to reconstruct the ultrasound image using the revised ultrasound information.

In accordance with yet other embodiments, a system is provided. The system includes an acquisition module including an ultrasound probe. The acquisition module is configured to acquire ultrasound information including anatomy information representative of a portion of anatomy to be imaged and artifact information representative of a coherent interference artifact that has been associated with a contrast agent. The ultrasound information includes information obtained by a plurality of readings along a single line. Due to association with the contrast agent, the coherent interference artifact appears in different realizations in the plurality of readings. The system also includes an analysis module including a processing unit. The analysis module is configured to receive the ultrasound information from the acquisition module and to suppress the artifact information by use of the plurality of readings along the single line. The system also includes a reconstruction module comprising a processing unit. The reconstruction module is configured to reconstruct an image using the revised ultrasound information from the analysis module.

### BRIEF DESCRIPTION OF THE DRAWINGS

FIG. 1 is a flowchart of a method for reconstructing an image in accordance with various embodiments.

FIG. 2 is a block diagram of an imaging system in accordance with various embodiments.

FIG. 3 is a flowchart of a method for reconstructing an image in accordance with various embodiments.

FIG. 4 depicts a group of RF waveforms corresponding to ultrasound readings taken along an A-line in accordance with various embodiments.

FIG. 5 depicts an ensemble average in accordance with various embodiments.

FIG. 6 is a block diagram of an exemplary ultrasound imaging system formed in accordance with various embodiments.

FIG. 7 is a block diagram illustrating a portion of the ultrasound imaging system shown in FIG. 6 in accordance with various embodiments.

FIG. 8 is a diagram illustrating a three-dimensional (3D) capable miniaturized ultrasound system in which various embodiments may be implemented.

FIG. 9 is a diagram illustrating a 3D capable hand carried or pocket-sized ultrasound imaging system in which various embodiments may be implemented.

FIG. 10 is a diagram illustrating a 3D capable console type ultrasound imaging system in which various embodiments may be implemented.

### DETAILED DESCRIPTION OF THE INVENTION

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or random access memory, hard disk, or the like) or multiple pieces of hardware. Similarly, the programs may be stand-alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

Also as used herein, the phrases “image” or “reconstructing an image” are not intended to exclude embodiments in which data representing an image is generated, but a viewable image is not. Therefore, as used herein the term “image” broadly refers to both viewable images and data representing a viewable image. However, many embodiments generate, or are configured to generate, at least one viewable image.

Various embodiments provide systems and methods for improved determination of parameters that may be used to reconstruct images and/or characterize tissue types using data obtained by ultrasound. For example, an ultrasound pulse is attenuated when propagating in biological tissues. Generally, a power law for the frequency dependence of the attenuation is defined which is tissue specific and influenced by the physical structure of the tissue. As the pulse propagates some of the energy is back scattered to a transducer where the pulse is recorded. As the frequency content of the pulse changes due to the frequency dependent attenuation of the tissue, the echoes generated from deeper in tissue have different frequency content than those produced more superficially. By analyzing the non-stationarity of the backscattered radio frequency (RF) waveforms, a first parameter, such as mean frequency, can be used to track this change. In some embodiments, taking the derivative of the first parameter (e.g. mean frequency) will provide a second parameter related to the rate of change of the first parameter. The second parameter, for example, may be used to reconstruct an image and/or to characterize a tissue type. Other mathematical techniques, such as spectral shift, may be employed to determine one or more parameters. Such parameters may be useful in providing diagnostic information, for example the identification of fibrous tissue or tumors. The presence of coherent interference artifacts that result from overlapping echoes (e.g. speckle), however, hampers the use of such approaches in practice with conventional ultrasound techniques. Various embodiments provide systems and methods for eliminating or reducing the effects of such artifacts to improve identification of parameters that may be useful in reconstructing images. Various embodiments provide system and methods for determining parameters related to tissue type at lower acoustic power settings than would be used by conventional systems.

For example, in some embodiments, a contrast agent is associated with a coherent interference artifact (e.g., speckle) so that the coherent interference artifact appears differently, or provides a plurality of different realizations, in readings taken along a single line at different times, allowing the coherent interference artifact to be identified and/or suppressed (e.g., all or a portion of the coherent interference artifact may be removed or reduced). The association of the contrast agent may provide detectable or identifiable spatial and/or temporal randomness in the coherent interference artifact that allow the coherent interference artifact to be suppressed by an averaging process. Thus, instead of appearing stationary as with conventional techniques, the coherent interference artifact is non-stationary, allowing for suppression of the coherent interference artifact.

A technical effect of at least one embodiment is improving image quality by suppressing or removing speckle or other coherent artifacts. Additionally, a technical effect of at least one embodiment is to allow determination of a parameter for parametric imaging that may not be discernible or reliably obtained using conventional ultrasound techniques. A further technical effect of at least one embodiment is allowing expanded use of ultrasound imaging, thereby reducing expense and/or improving access to medical imaging.

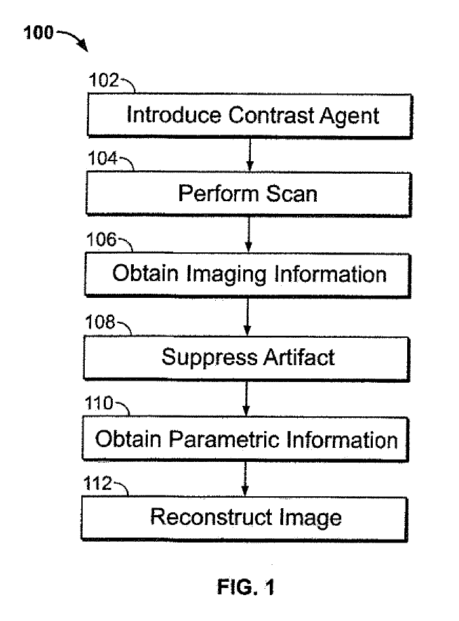

FIG. 1 is a flowchart of a method 100 for reconstructing an image in accordance with various embodiments. Systems and methods in accordance with various embodiments, such as the method 100, for example, employ the use of a contrast agent to improve identification of artifacts, such as coherent interference artifacts, and removal or suppression of artifacts in reconstructed images. For example, speckle is an artifact encountered in ultrasound imaging that results from overlapping echoes. Artifacts such as speckle may appear as stationary in a conventional ultrasound image, and thus may not be distinguishable from anatomical structures or tissues. A tissue plane imaged using conventional techniques may appear to have a stable speckle pattern. By associating contrast agent with the artifact in accordance with some embodiments, the speckle may be made to appear non-stationary, so that the artifact may be identified. For example, random scatters may be introduced that destroy or reduce the temporal coherence of the artifact and facilitate suppression of the artifact. In accordance with various embodiments, contrast agents may be employed, with a contrast agent imaging mode, such as pulse inversion, resulting in an a observable speckle pattern that continually changes because the contrast agents are in the blood pool and moving.

Using conventional techniques, a coherent interference artifact such as speckle may appear to be stationary at standard or conventionally employed acoustic power levels. In accordance with various embodiments, however, the coherent interference artifact may be made to appear non-stationary in readings taken at different times by introducing random (temporal and spatial) scatters such as moving contrast agent bubbles. After the coherent interference artifact is made to appear non-stationary in the image, the coherent interference artifact may be identified and/or removed from an image. For example, a plurality of readings may be taken along a single line at different times, providing different realizations of speckle pattern due to the use of the contrast agent. The different realizations of speckle may then be used to average away the effect of speckle. In various embodiments, ten or more RF waveforms may be obtained along each A-line (e.g., a line of sight from a particular measurement location) of a scan.

In various embodiments, certain steps may be omitted or added, certain steps may be combined, certain steps may be performed simultaneously, or concurrently, certain steps may be split into multiple steps, certain steps may be performed in a different order, or certain steps or series of steps may be re-performed in an iterative fashion. The method 100 may be performed, for example, in association with systems such as those discussed herein.

At 102, a contrast agent is introduced into the object, such as a patient. The contrast agent may be introduced by injection of a bolus intravenously into a patient. For example, a contrast agent, such as, for example, Optison™ (Perflutren Protein-Type A Microspheres Injectable Suspension), or, as another example, Sonazoid™, may be introduced into the patient. In various embodiments, the contrast agent is associated with the coherent interference artifact but is not substantially associated with the portion of anatomy to be imaged. For example, a scan may be performed to provide an image of the liver of a patient. The contrast agent may then be introduced into the blood stream so that the contrast agent may be used to strengthen the echo of blood flow surrounding the portion of the anatomy being imaged, providing a non-stationary appearance of speckle in realizations acquired at different times, without substantially enhancing or strengthening echoes associated with the tissue of the liver. A relatively low dose of contrast agent may be employed so that the artifact is made to appear non-stationary while still not substantially affecting tissue attenuation of a portion of the body being imaged.

Various embodiments do not use such contrast agents to provide a contrast-enhanced image (e.g. allowing easier distinguishing of blood from surrounding tissue in a reconstructed image), but instead use the modified echo of aspects surrounding the tissue of interest to suppress artifacts. Thus, in some embodiments, all or a portion of the data corresponding to one or more coherent interference artifacts is removed from the data set used to reconstruct the image, and the tissue of interest (in this example, tissue of the liver) is more accurately rendered. For example, if an image of the liver or a portion of the liver is desired, the contrast agent may be associated with a blood pool of the patient, but not substantially associated with the liver. Thus, a background artifact may be associated with a contrast agent for improved detection and removal of the artifact (e.g., by averaging away different realizations of the artifact at different data acquisition times). For example, in some embodiments, substantially all of the imaging information that has been associated substantially with a contrast agent is removed before the image is reconstructed. As another example, in some embodiments, a majority of the imaging information that has been associated with a contrast agent is removed before the image is reconstructed.

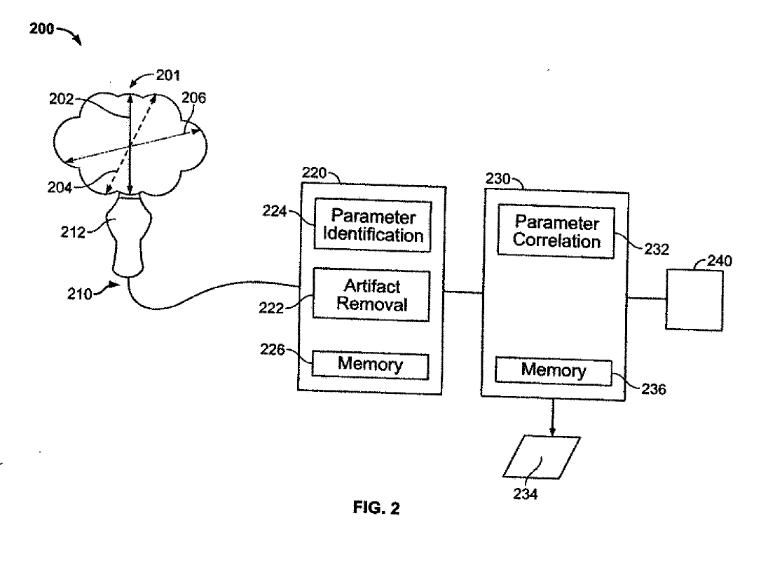

At 104, a scan is performed. For example, an ultrasound scan may be performed on aspect region of interest of a patient using an imaging system including an ultrasound transceiver. An example of such a system is shown in FIG. 2.

FIG. 2 illustrates a block diagram of an imaging system 200 in accordance with various embodiments. In FIG. 2, the imaging system 200 is depicted as being used to provide an image of an object 201, for example tissue of a patient. In the illustrated embodiment, the imaging system 200 includes an acquisition module 210, an analysis module 220, a reconstruction module 230, and a display module 240. In the illustrated embodiment, the imaging system 200 is configured to perform a scan of an object, for example an aspect or region of interest of a patient, and to acquire imaging data during the scan. The imaging data acquired during the scan may then be transmitted to the analysis module 220, where one or more parameters are identified. Imaging information including parametric information corresponding to the identified parameter(s) may then be transmitted to the reconstruction module 230, where an image representing the object scanned is reconstructed using at least the parametric information communicated from the analysis module 220. The display module 240 is configured to present a viewable image or otherwise provide access to a reconstructed image from the reconstruction module 230 for viewing the parametric and/or morphologic information, and/or for further analysis.

The acquisition module 210 is configured to acquire scanning information. For example, in the illustrated embodiment the acquisition module 210 includes an ultrasound transceiver 212. The ultrasound transceiver may include a transmitter that drives an array of elements (e.g., piezoelectric elements) within a probe to emit pulsed ultrasonic signals or waves, into a body. The ultrasonic signals or waves are back-scattered from structures in the body, to produce echoes that return to the transceiver 212. The echoes, for example, may be received by a receiver of the transceiver. The received echoes may then be used to output an RF signal.

In the illustrated embodiment, the acquisition module 210 is used to acquire information representative of the object 201, such as a portion of a patient. For example, in the illustrated embodiment, the acquisition module is configured to collect data at a plurality of pulse-echo-measurement locations. For example, data may be collected by a practitioner at a first measurement location located along an outer edge of the object 201, with the data collected along a first line 202. Collection and subsequent reconstruction along a single line (an “A-Line”) may be referred to as an “A-mode” of ultrasound. The transceiver may be positioned to a second position along the outer edge of the object 201, with data collected along a second line 204 (depicted with a dashed line in FIG. 2). The transceiver may be subsequently re-positioned and data taken along a line corresponding to each position up to an “n”th line 206 (depicted with a phantom line in FIG. 2), with the lines being generally co-planar. Imaging information from each of the lines (e.g. first line 202, second line 204, . . . , nth line 206) may then be summed to provide an image of a plane including the A-lines 1, 2, . . . n. The reconstruction of an ultrasound image in a plane may be referred to as the “B-mode.” For example, in some embodiments, 100 or more A-lines may be used to reconstruct a planar image.

The analysis module 220 is configured to analyze imaging information and to determine one or more parameters based upon the imaging information. For example, in the illustrated embodiment, the analysis module 220 receives imaging information from the detection module 210, removes artifacts from the received scanning information, and determines a parameter for image reconstruction using the scanning information with the artifacts removed. The received information may be considered as including a first portion corresponding to the structure, tissue, or region of interest desired to be imaged and a second portion corresponding to artifacts. To provide improved imaging, all or a portion of the second portion may be removed or suppressed before reconstructing the image. In the illustrated embodiment, the analysis module includes an artifact removal module 222, a parameter identification module 224, and a memory 226.

The artifact removal module 222 is configured to remove or suppress an artifact (e.g., a coherent interference artifact that has been associated with a contrast agent) from imaging information. For example, in the illustrated embodiment, the artifact removal module 222 is configured to receive imaging information (for example, imaging information acquired by the acquisition module 210), identify and/or remove an artifact (e.g. speckle) from the imaging information to provide revised or updated imaging information, and to provide the revised or updated imaging information to the parameter identification module 224. For example, the artifact removal module 222 may be configured to receive a set of waveforms representing readings taken along a single given A-line, and combine the set into a composite or ensemble waveform that averages out speckle and/or other artifacts or otherwise removes or accounts for the speckle and/or other artifacts. For example, in various embodiments, the artifact removal module 222 may receive information from a plurality of readings taken along a given A-line. The artifact removal module 222 may then organize the readings into pairs, and subtract one member of each pair from the other member of the pair to form a differenced pair. The differenced pairs may then be averaged to form an ensemble waveform, with a portion or all of the effects of speckle averaged out.

The parameter identification module 224 is configured to determine one or more parameters based on received imaging information (e.g. raw and/or processed imaging information). For example, in the illustrated embodiment, the parameter identification module 224 is configured to receive revised imaging information (e.g. imaging information with speckle removed) from the artifact removal module 222, and determine one or more parameters that may subsequently be utilized by the reconstruction module 230 to reconstruct an image. For example, the imaging information obtained by the parameter identification module 224 may include information corresponding to the amplitude of an RF waveform (e.g. an ensemble waveform) for a given A-line at various frequencies. The one or more parameters obtained may include, for example, a mean frequency that tracks the change of the frequency content of a pulse due to frequency dependent attenuation of tissue through which the pulse has passed. As another example, the one or more parameters may also include a derivative of such a frequency that is related to the rate of change of the above discussed frequency content.

The memory 226 is operably connected to all or some of the other components of the imaging system 200 including aspects of the analysis module 220 such as the artifact removal module 222 and the parameter identification module 224, and is configured to store data for use by the other modules and/or users of the imaging system 200. For example, the memory 226 may store scanning information provided by the acquisition module 210, revised scanning information provided by the artifact removal module 222, or parametric information determined by the parameter identification module 224.

The reconstruction module 230 is configured to reconstruct an image using imaging information, for example parametric information provided by the analysis module 220. For example, in the illustrated embodiment, the reconstruction module 230 receives parametric information from the analysis module 220 (e.g. the parameter identification module 224 of the analysis module) and reconstructs an image using the parametric information. The reconstructed image, for example, may be viewable on the display module 240 (for example, on a touchscreen), or, as another example, the reconstruction module 230 may output data 234 representative of the reconstructed image. The data 234 may be provided for further processing, or, as another example, may be provided to a medical data storage system. In the illustrated embodiment, the reconstruction module 230 includes a parameter correlation module 232 and a memory 236.

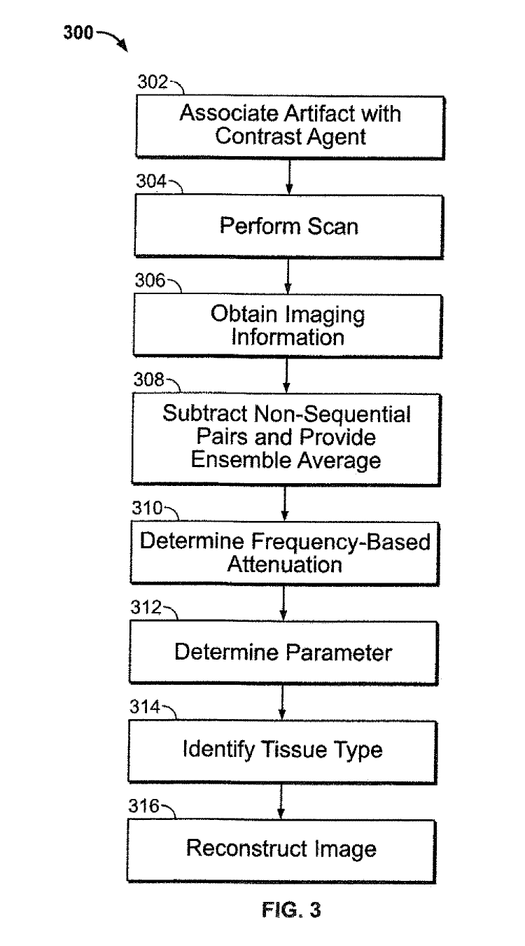

The parameter correlation module 232 receives parametric information from the analysis module, and, for example, determines a tissue type using the parametric information. For example, the parametric information may include information describing, depicting, or corresponding to a given parameter along a length of an A-line. The parametric correlation module 232 then determines the tissue type or types along the length of the A-line. For example, the parametric correlation module 232 may access a look-up table that lists tissue types corresponding to values or ranges of values of one or more parameters. For example, in some embodiments a parameter β may be determined by the parameter identification module of the analysis module 220. The parameter β describes the frequency dependence of the amplitude of a signal through a type of tissue, and may be used to differentiate one type of tissue from another type of tissue. (See also discussion related to FIG. 3.) For example, the value of β for fat tissue is different than the value of β for fibrous tissue.

After the type of tissue has been identified using the parameter correlation module 232, a parametric image may be reconstructed by the reconstruction module 230. For example, a series of A-lines may be analyzed, with the type of tissue along each A-line determined, and then each A-line individually reconstructed. Then, the individual A-line reconstructions may be added together to form a planar image that has been reconstructed using imaging information from the acquisition module 210.

The memory 236 is operably connected to all or some of the other components of the imaging system 200 including aspects of the reconstruction module 230 such as the parameter correlation module 232, and is configured to store data for use by the other modules and/or users of the imaging system 200. For example, the memory 236 may store parametric information received from the parameter identification module 224, or a reconstructed image or related data determined by the reconstruction module 230.

Returning to FIG. 1, at 104, as indicated above, a scan is performed. The scan may be performed, for example, using an acquisition module such as the acquisition module 210 discussed above to transmit an ultrasound beam to an aspect or region of interest of a patient. For example, ultrasound information or measurements may be acquired or determined along a series of A-lines, such as 202, 204, 206 (see FIG. 2). In some embodiments, plural sets or readings of imaging information may be acquired along each A-line. For example, in some embodiments, ten readings may be taken along each of the first through nth lines 202, 204, . . . 206. More readings or fewer readings may be taken along one or more A-lines in other embodiments. The information acquired during the scan is collected and forwarded for subsequent processing and reconstruction of an image.

Each reading along a given A-line may correspond to more than one pulse sent by an ultrasound transceiver. For example, in pulse inversion imaging, two pulses are sent in rapid succession into the object 201, with the second pulse a mirror image of the first pulse. The resulting echoes are added at reception. When the resulting echoes are added, linear scattering (which dominates in tissues) may be cancelled out, as linear scattering may provide two echoes that are inverted copies of each other. Non-linear scattering (which may result from gas microbubbles, such as microbubbles associated with contrast agents) may not be cancelled out, thus strengthening the appearance of aspects associated with the contrast agents.

At 106, imaging information is obtained. For example, in some embodiments, imaging information is obtained by a processing unit, such as the analysis module 220, from an acquisition module 210 as discussed above. The imaging information may be obtained in a raw form, or in a form that has been partially processed (for example to have noise removed). The imaging information in some embodiments is received as a series of RF A-line waveforms. For example, a processing unit may obtain a series of ten waveforms taken at each pulse-echo location defining a B-mode plane. The imaging information may be considered as being composed of two types of information that have been combined including a first type of information corresponding to a structure or tissue of interest for which it is desired to reconstruct an image, and a second type of information corresponding to coherent artifacts such as speckle. Other types of information, such as non-coherent artifacts may also be present, and/or may be addressed by additional processing techniques as will be appreciated by one having ordinary skill in the art.

At 108, the artifact is removed or suppressed. The removal or suppression of the artifact (or imaging information caused by or associated with the artifact or the source of the artifact) may be performed, for example, by a processing unit including an analysis module such as the analysis module 220, including, for example, an artifact removal module 222. In some embodiments, the imaging information may be grouped together by A-line and, in some embodiments, the groups for each A-line may be further combined or processed to average out or otherwise remove scanning information corresponding to an artifact. For example, in some embodiments, the series of waveforms for a given A-line are grouped into pairs, with one member of each pair subtracted from the other member of the pair to form a differenced pair. The differenced pair for the given A-line may then be combined or averaged to form an ensemble waveform for the given A-line with all or a portion of an artifact such as speckle removed.

At 110, parametric information is obtained. For example, parametric information may be obtained by a processing unit such as the analysis module 220, for example, including a parameter identification module 224, as discussed above. The parametric information may be obtained, for example, by analyzing imaging information, such as revised imaging information having effects of speckle removed or suppressed that is provided by, for example, a processing unit such as the artifact removal module 222 discussed above. In some embodiments, the parametric information includes a first parameter corresponding to a frequency attenuation of a waveform in a tissue of interest, and a second parameter corresponding to a rate of change with respect to frequency of the first parameter.

At 112, an image is reconstructed. The image may be reconstructed, for example, by a processing unit such as the reconstruction module 230. For example, parametric information may be used to identify a type or types of tissue along a given A-line, and an image for the given A-line reconstructed accordingly. Subsequent A-lines may be similarly reconstructed individually. The reconstructed A-line images may then be combined to provide a B-mode planar reconstructed image. In some embodiments, the planar images may in turn be combined to provide a three dimensional image.

FIG. 3 is a flowchart of a method 300 for reconstructing an image in accordance with various embodiments. The method 300, for example, employs the use of a contrast agent to improve identification of artifacts and removal or suppression of artifacts from imaging data. In various embodiments, certain steps may be omitted or added, certain steps may be combined, certain steps may be performed simultaneously, or concurrently, certain steps may be split into multiple steps, certain steps may be performed in a different order, or certain steps or series of steps may be re-performed in an iterative fashion. The method 300 may be performed, for example, in association with systems such as those discussed herein.

At 302, an artifact is associated with a contrast agent. For example, a contrast agent may be introduced into an object to be scanned, such as a patient. The contrast agent may be introduced by injection of a bolus intravenously into a patient. For example, in some embodiments, a scan may be performed to provide an image of the liver of a patient. The contrast agent may then be introduced into the blood stream so that the contrast agent may be used to strengthen the echo of blood flow surrounding the portion of the anatomy being imaged. The background artifact may be associated with the contrast agent to provide a non-stationary appearance of the artifact over time for improved detection of the artifact, which in turn allows for improved removal of the artifact.

At 304, a scan is performed. For example, an ultrasound scan may be performed on aspect region of interest of a patient using an imaging system including an ultrasound transceiver. For example a series of signals may be sent and received along a given A-line from a pulse echo measurement position. The pulse echo measurement position may be changed to allow sending and receiving of a subsequent series of signals along a second A-line. The process may be repeated for a number of different A-lines. Information from the plurality of A-lines may be subsequently combined to provide a planar image.

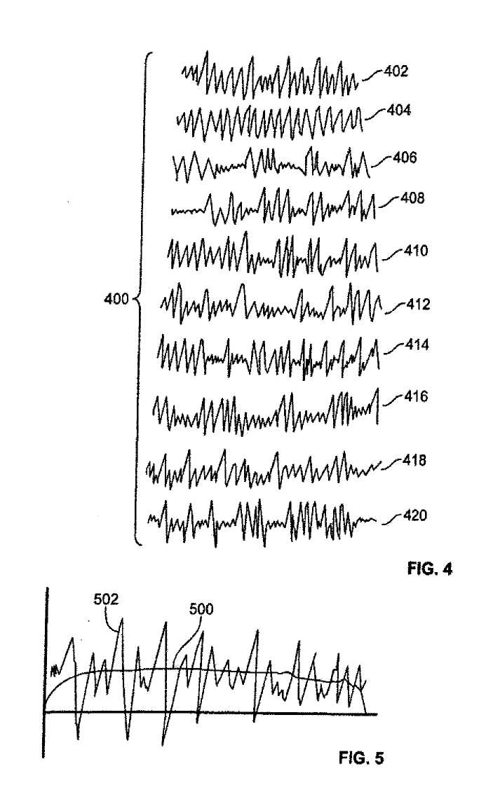

At 306, imaging information is obtained. The imaging information may be obtained, for example, by a processing unit configured to receive imaging information from a detector such as an ultrasound transceiver. The processing unit may also be configured to remove or suppress artifact information from the imaging information and to determine one or more parameters of an object being scanned. For example, ten data sets or readings for each A-line of an ultrasound scan may be obtained. FIG. 4 depicts a group of RF waveforms 400 corresponding to ultrasound readings taken along an A-line. The data sets or readings for a given A-line may be organized or grouped as depicted in the below table. In the illustrated embodiment, the readings are taken about 25 milliseconds apart. FIG. 4 depicts ten sequentially obtained RF signals taken along a given A-line of an ultrasound scan (RF signals). The table below summarizes the times at which the RF A-line signals are obtained in the depicted embodiment.

| | Position in Sequence of | |
|---|---|---|
| | Acquired A-line Signals | |
| Reference Number | (Sequence Number) | Time at which obtained |
| 402 | 1 (rf1) | T₀ = 0 |
| 404 | 2 (rf2) | T₀ + 25 milliseconds |
| 406 | 3 (rf3) | T₀ + 50 milliseconds |
| 408 | 4 (rf4) | T₀ + 75 milliseconds |
| 410 | 5 (rf5) | T₀ + 100 milliseconds |
| 412 | 6 (rf6) | T₀ + 125 milliseconds |
| 414 | 7 (rf7) | T₀ + 150 milliseconds |
| 416 | 8 (rf8) | T₀ + 175 milliseconds |
| 418 | 9 (rf9) | T₀ + 200 milliseconds |
| 420 | 10 (rf10) | T₀ + 225 milliseconds |

As also indicated above in connection with the discussion of FIGS. 1 and 2, the imaging information may be considered as including two different components or subsets. A first subset may be defined as imaging information from stationary structures (e.g. tissue being imaged), and a second subset may be defined as imaging information from artifact sources, for example a coherent interference artifact such as speckle. An improved image may be achieved by extracting the portion of the signal corresponding to the tissue of interest from the entire signal (e.g. the signal including the information regarding the tissue being imaged and also including the artifact imaging information). For example, an RF A-line signal, f(t), acquired for a given A-line (e.g. lines 202) can be modeled as being comprised of two components. One component is from the stationary structures in the tissue, fₛ(t), and one component is from the contrast agents that are flowing in the blood pool, f_{ca}(t). This may be described as:

f(t)=fₛ(t)+f_{ca}(t) (1)

The tissue may be probed at different times. For example, in some embodiments 10 data sets are acquired at each A-line. During the time interval between data set acquisition along a given A-Line, the contrast agent has moved with the blood and the echoes from the contrast agent at the two times will be shifted (as the contrast agent is moving) and decorrelated (as some of the contrast agent moves out of the volume being probed, and is replaced by other contrast agent). Subtraction of the two waveforms (one taken at a first time, and one taken at a second time) may effectively remove the component from the image of the structure (e.g. the tissue being imaged). If the time interval is long enough for decorrelation of the agent signal to take place, then the result of the subtraction represents scattering from the contrast agent.

f_{d}=f₁(t)−f₂(t)=fₛ₁(t)+f_{ca1}(t)−fₛ₂(t)−f_{ca2}(t)≈f_{ca1}(t)−f_{ca2}(t) (2)

Production of a difference signal measured this way may be achieved, for example, using pulsed Doppler techniques, as will be appreciated by one having ordinary skill in the art. The extraction of the contrast agent specific portion of the signal may also be obtained from other techniques known by those skilled in the art, such as pulse inversion or pulse phase sequences. The use of contrast agents allows measurement with a higher signal to noise ratio (SNR) and much greater sensitivity than scattering from blood alone. Thus, fundamental scatter from tissue scattering by nature of the decorrelation and movement of the contrast agent may be observed or identified and extracted, allowing the use of lower acoustic powers than would otherwise be necessary.

Sufficient decorrelation of the of the contrast agent signal may be useful to stop distortion of the scattering spectra of the differenced contrast agent contributions. For example, consider the limit of two spectra taken differenced with very little movement and decorrelation. The contrast agent components will be highly correlated and experience a small shift (in time, δt, between the echo sequences). If the limit is

$${lim}_{δ t -> 0} \frac{f ( t ) - f ( t - δ t )}{δ t} = {f}^{′} ( t ) ,$$

it can be seen that the difference relates to the derivative of the contrast agent contribution. Thus, the spectrum will be distorted by a weighting of −i2πf in the frequency domain. Therefore, measurements may be separated by a sufficient time, for example, about 100 ms, to allow for sufficient decorrelation. For example, with reference to FIG. 4 and the corresponding table, if RF waveforms are acquired from a particular pulse-echo line of sight (e.g. 402, 404, 406, 408, 410, etc.) about every 25 milliseconds, then the difference may be performed between rf1 1 (taken at a t₀ of about 0) and rf6 (taken about 125 milliseconds later), rf2 (taken at a time of about 25 milliseconds) and rf7 (taken at a time of about 150 milliseconds (about 125 milliseconds after rf2), etc. (see FIG. 4). By differencing non-sequential pairs, (e.g. rf1 and rf6) instead of pairs of waveforms obtained at adjacent time periods (e.g. rf1 and rf2), a greater time interval is provided between the pair of waveforms differenced to provide a given ensemble, thereby providing for increased decorrelation between the corresponding time intervals of the differenced pairs.

At 308, as also discussed above, non-sequential pairs of RF waveforms are subtracted to provide ensemble sets that are averaged to provide an ensemble average. As discussed above, non-sequential pairs may be differenced to provide adequate time between the pair members to allow decorrelation. Thus, for example, a differenced pair rfd1 (or ensemble set) may be obtained by subtracting rf6 from rf1, a differenced pair rfd2 may be obtained by subtracting rf7 from rf2, and so on. In some embodiments, differenced RF A-line pairs produce the contrast agent specific signal and provide the ensemble sets for averaging away the coherent interference artifacts. The procedure may be repeated at each pulse-echo measurement position, and the 1D measurements stacked together to form a 2D image. Data acquisition time for some applications is about 1-3 seconds. FIG. 5 depicts an ensemble average 500 in accordance with various embodiments. In the illustrated embodiment, the ensemble average 500 represents a signal formed by averaging differenced pairs, such as differenced pairs rfd1, rfd2, etc. formed from the RF waveforms of FIG. 4. Waveform 502 represents a single realization of an RF waveform that still includes, for example, effects of speckle and/or other artifacts.

At 310, a frequency-based attenuation characteristic or characteristics of the tissue of interest is determined. For example, a signal or signals provided at 308 (e.g. one or more ensemble averages such as ensemble average 500) may be analyzed to determine attenuation characteristics of the tissue being scanned by a given RF A-line signal. The frequency based attenuation characteristics of the tissue may be used to determine one or more parameters of the tissue being scanned.

For example, an RF A-line pulse echo sequence may be examined in terms of Entire Function Theory (EFT). EFT may provide a presentation of amplitude and phase relationships (particularly coherent interference artifacts) in a particularly revealing manner. To aid the reader's understanding, some background is also presented herein for instantaneous frequency (IF) and the nature of large excursions that may occur in the analysis of RF waveforms and the link to coherent interference artifacts (such as speckle), along with the link to zeros of the EFT model in the complex time and frequency planes.

Because digital data by definition is strictly band limited, RF A-line signals are strictly band limited, in the sense that, F(ω)=0 for |ω|>σ, where F(ω) is the Fourier Transform of f(t), the RF A-line signal, and σ is finite. Given the strictly band limited nature, the associated analytic signal, s(t), can be defined via,

s(t)=f(t)+HT{f(t)} (3)

where i=\sqrt{−1}, and HT{f(t)} is the Hilbert Transformation of f(t) defined as,

$$HT \{ f ( t ) \} = \frac{1}{π} p . v . {\int }_{- ∞}^{∞} \frac{f ( τ )}{( t - τ )} d τ = f ( t ) ⊗ \frac{1}{π t} . \qquad ( 4 )$$

[symbol]

denotes the convolution operator and p.v. the Cauchy Principle value of the integral (to accommodate the divergence at t=τ). The envelope a(t), phase φ(t) and instantaneous frequency Φ(t) can be defined in terms of the analytic signal via,

$$a ( t ) ≡ \vert s ( t ) \vert = {\{ {f}^{2} ( t ) + {HT [ f ( t ) ]}^{2} \}}^{\frac{1}{2}} \\ ϕ ( t ) ≡ arg [ s ( t ) ] = arctan [ \frac{HT \{ f ( t ) \}}{f ( t )} ] \\ Φ ( t ) ≡ \frac{1}{2 π} \frac{d ϕ ( t )}{d t} = \frac{f ( t ) {HT}^{′} \{ f ( t ) \} - HT \{ f ( t ) \} {f}^{′} ( t )}{{f}^{2} ( t ) + {HT}^{2} \{ f ( t ) \}} \qquad ( 5 )$$

The analytic signal s(t) may be expressed in modulus phase form, s(t)=a(t) e^{iφ(t)}. It should be noted that the strictly band limited nature of s(t) leads to a strict band limited nature of a²(t), with up to twice the bandwidth of s(t), but not to a(t). The phase function may also contain discontinuities and therefore is not necessarily strictly band limited.

The instantaneous frequency (IF) may also be described as the first conditional moment (in frequency) of any time-frequency distribution that satisfies the marginals (such as the Wigner or Choi-Williams distribution), by definition.

Next, attention is turned to analytic properties of a complex pulse-echo signal, such as a pulse-echo signal obtained directly or indirectly from an ultrasound transducer as discussed above. For example, let zₜ=τ+iσ define a complex variable. The analytic signal can be continued into the complex time plane via analytic continuation via the Fourier Transformation,

s(zₜ)=·₀^{∞}S(f)e^{i2πz}^{ₜ}df=·₀^{∞}S(f)e^{-2πfσ}e^{2πifτ}df (6)

The above is convergent for all σ≧0 and is therefore analytic in the upper half plane. It follows from the strict band limited nature that it is analytic also in the finite lower half plane. These properties give rise to the nomenclature “analytic signal.”

The relationship between the instantaneous or time domain frequencies and cousins thereof in the Fourier domain may not appear obvious. There is, however, a relationship between the appropriately weighted moments. For RF A-line segments of arbitrary length L centered at τ,

$${\overline{f}}_{τ} = \frac{{\int }_{0}^{∞} f {\vert {S}_{τ} ( f ) \vert}^{2} d f}{{\int }_{0}^{∞} {\vert {S}_{τ} ( f ) \vert}^{2} d f} = \frac{1}{2 π} \frac{{\int }_{τ - \frac{L}{2}}^{τ + \frac{L}{2}} Φ ( t ) {a}^{2} ( t ) d t}{{\int }_{τ - \frac{L}{2}}^{τ + \frac{L}{2}} {a}^{2} ( t ) d t} \qquad ( 7 )$$

This result may be used for the efficient calculation of parametric images. From the above it may, seem likely that the variance associated with the IF would be greater than that associated with the energy density spectrum. However, the opposite may be shown. As a consequence, large IF excursions from the mean are restricted or ‘concentrated’ in time.

In practice, the signals may be manipulated in discrete form. The following estimator for determining the IF from sampled data (the central finite difference method) may be employed. The IF may be point-wise recoverable from a zeros representation of the discrete waveform. However, for ease of calculation, the form below may be used.

$$Φ [ n ] = \frac{{f}_{s}}{4 π} \{ arg ( s [ n + 1 ] ) - arg ( s [ n - 1 ] ) \} mod .2 π \qquad ( 8 )$$

where fₛ is the sampling frequency and mod·2π is modulo 2pi.

The discrete representation may be expressed as a finite Fourier Series,

f(zₜ)=Σₖ₌ₙ_{₁}ⁿ^{₂}Cₖe^{ikΩz}^{ₜ} (9)

where n₁≦n₂ and the fundamental frequency Ω=1/nT, and where T is the sampling period. The periodic nature of f(zₜ) implies that f(zₜ) is characterized completely by the values f(zₜ) takes in any z-plane strip of width 2π/Ω which is parallel to the imaginary axis. Hence a trigonometric polynomial in the variable zₜ may be represented by an algebraic polynomial in the variable w by means of the mapping,

w=e^{iΩz}^{ₜ} (10)

Hence it may be derived,

$$f ( {z}_{t} ) = {c}_{{n}_{2}} {w}^{{n}_{1}} {\sum }_{k = {n}_{1}}^{{n}_{2}} ( \frac{{c}_{k}}{{c}_{{n}_{2}}} ) {w}^{k - {n}_{1}} \qquad ( 11 )$$

The summation is a polynomial of n_{d}=n₂−n₁, and has n_{d} roots denoted by the sequence w₁, . . . , wₙ. A unique set of zeros, {zₖ} may then be defined via the relation,

wₖ=e^{iΩz}^{ₖ} (12)

The product expansion for f(zₜ) is then given by the expansion,

f(zₜ)=Cₙ_{₂}eⁱⁿ^{₁}^{Ωz}^{ₜ}Πₖ₌₁ⁿe^{iΩz}^{ₜ}−e^{iΩz}^{ₖ} (13)

Next, attention is turned to the zero representation of phase envelope relationships. If zₜ=zₙ denotes the location of a zero of s(zₜ) of order rₙ, then it can be shown that,

$$Φ ( t ) = {k}_{1} + {\sum }_{n} \frac{{r}_{n} {σ}_{n}}{{( t - {τ}_{n} )}^{2} + {σ}_{n}^{2}} \\ {k}_{1} = Φ ( 0 ) - {\sum }_{n} \frac{{r}_{n} {σ}_{n}}{{\vert {z}_{n} \vert}^{2}} \\ {k}_{2} = {ln}^{′} a ( 0 ) + {\sum }_{n} \frac{{r}_{n} {τ}_{n}}{{\vert {z}_{n} \vert}^{2}} \qquad ( 14 )$$

These results are valid for any strictly band limited function and are therefore widely applicable.

The reason for the large excursions in instantaneous frequency often seen in RF waveforms can be related directly to the presence of a single zero close to the real time axis. Consider the contribution from a zero of order rₙ located at zₙ=τₙ+iσₙ. As t goes from τₙ−ε to τₙ+ε, ln′ a(t) goes from negative to positive, thus encoding an envelope minimum at t=τₙ. The IF, Φ(t), goes through a maximum deviation of rₙ/σₙ at t=τₙ, which may tend to infinity as σₙ tends to 0.

EFT thus provides a convenient description of many properties of RF waveforms. The zero description in the complex time plane provides an intuitive description of the distortion of envelope and phase features of the RF waveform by coherent interference artifacts such as speckle. Destructive interference effects can be produced that reduce the envelope signal (even to 0) and produce a corresponding phase distortion (which may approach +/−infinity). Thus, large ‘noise’ terms present in an IF determined from information obtained and used with conventional ultrasound techniques may be caused by coherent interference effects.

The concept of an instantaneous bandwidth, or 1B, can also be defined (note that the bandwidth appears as the f̅²̅term in the parametric imaging discussion below). It can be expressed in terms of the zeros as,

$${IB}^{2} ≡ {( \frac{{a}^{′} ( t )}{a ( t )} )}^{2} = {[ {k}_{2} + {\sum }_{n} ( \frac{{r}_{n} {a}_{n}}{{( t - {τ}_{n} )}^{2} + {σ}_{n}} \cdot \frac{( t - {τ}_{n} )}{{σ}_{n}} ) ]}^{2} \qquad ( 15 )$$

Attention will now be turned to the calculation of a set of complex time domain zeros. First, the (discrete) waveform may be Fourier transformed. The time domain zeros may be defined as the set of roots from the Fourier coefficients that span the spectral bandwidth of the signal. The time domain zeros can be efficiently computed, for example, by finding the eigenvalues of the associated companion matrix (transforming the problem into eigenvalue calculation).

Next, attention is turned to determination of parameters using rf A-line information as discussed above. A common model for the intensity of a monochromatic plane wave travelling in the positive x direction into a uniform isotropic tissue sample is as follows:

I(x,f)=I(x₀,f)e^{-μ(f)[x-x}^{₀}^{]} (16)

where x is the frequency of the wave and I(x₀,f) is the intensity at some reference point. μ(F) is the frequency dependent attenuation coefficient. Similarly an amplitude attenuation coefficient may be represented as follows:

A(x;f)=A(x₀;f)e^{-α(f)[x-x}^{₀}^{]} (17)

with μ(f)=2α(f). A model for α(F) (in dB/cm) for about 1-10 MHz is as follows:

α(f)≈βf (18)

Thus, α (the frequency-dependent amplitude attenuation) is proportional to the frequency by the parameter β. The higher β is, the stronger correlation there is between amplitude and frequency, or the more frequency dependent amplitude is. The parameter β may be used to differentiate one type of tissue from another type of tissue. For example, the value of β for fat tissue is different than the value of β for fibrous tissue. Thus, in some embodiments, frequency and amplitude (along with the dependency of amplitude on frequency, or the frequency dependent attenuation) are determined along one or more A-line samples, and used to determine β, which in turn may be used to identify a type or types of tissue. In some embodiments, the use of techniques described herein to remove or suppress artifacts from acquired imaging information allows for improved determination of one or parameters such as β.

For example, for a pulse amplitude spectrum, P(f;x) propagating in an inhomogenous medium the following approximation may be used:

α(x,f)=α₀(x)f (19)

Further, P(f;x) may be expressed as

$$P ( f ; x ) = P ( f ; {x}_{0} ) {e}^{[ - {\int }_{{x}_{0}}^{x} {α}_{0} ( {x}^{′} ) d {x}^{′} \cdot f ]} \qquad ( 20 )$$

The gradient of the mean frequency (after some algebraic manipulation) may be given by the following:

$$\frac{d \overline{f}}{d x} ≡ \frac{d}{d x} [ \frac{{\int }_{- ∞}^{∞} {fP}^{2} ( f ; x ) d f}{{\int }_{- ∞}^{∞} {P}^{2} ( f ; x ) d f} ] = - 2 {α}_{0} ( x ) \\ [ \frac{{\int }_{- ∞}^{∞} {f}^{2} {P}^{2} ( f ; x ) d f}{{\int }_{- ∞}^{∞} {P}^{2} ( f ; x ) d f} - \frac{{\{ {\int }_{- ∞}^{∞} {fP}^{2} ( f ; x ) d f \}}^{2}}{{\{ {\int }_{- ∞}^{∞} {P}^{2} ( f ; x ) d f \}}^{2}} ] = -- 2 {α}_{0} ( x ) \overline{{f}^{2}} \\ ⇒ {α}_{0} ( x ) = \frac{d \overline{f}}{d x} \frac{1}{2 \overline{{f}^{2}}} \qquad ( 21 )$$

For Gaussian pulses, f̅²̅ is constant with the assumed linear frequency dependent attenuation model. Alternatively f̅²̅ may be estimated via the instantaneous bandwidth (Equation (15)). α is an example of a frequency based attenuation characteristic that may be used to determine a parameter (e.g. β) that may in turn be used to determine a tissue type.

One method of computation of the mean frequency is provided by Equation (7). The RF-Aline signal is Hilbert transformed and the envelope and instantaneous frequency may be calculated and multiplied. The function may then be windowed (alternatively, block processing may be employed, or the entire signal convolved with the window function). The same procedure block or filtering procedure may be applied to the envelope function and a simple division yields the mean frequency estimate. It should be noted that such a model may only work for the fundamental, and may need to be modified if harmonic imaging signals are being used in order to extract bubble specific echoes, and to obtain a direct relationship to an attenuation coefficient.

In other embodiments, frequencies may be determined by alternative methods or techniques. For example, the spectral shift method provides an alternative to using instantaneous frequency, or mean frequency, tracking. For example, in some embodiments employing a spectral shift method, the RF A-line signal may be assumed as piecewise stationary, using the following:

S(f;x)=P(f;x)·T(f;x) (22)

Here, T(f, x) is the amplitude spectrum of the tissue (back) scattering function. The parameter x indicates the mid-point of the data segment extraction window. The pulse amplitude spectrum of segment j can be related to that of segment i (separated by a distance d) via,

$$P ( f ; x ) = P ( f , {x}_{i} ) {e}^{- 2 {\int }_{{x}_{i}}^{{x}_{j}} {α}_{0} ( {x}^{′} ) d {x}^{′} f} \qquad ( 23 )$$

Equation (22) implies that the estimate \hat{x}ᵢⱼ of the amount of attenuation experienced by the pulse when propagating from xᵢ to xⱼ may be estimated from the pulse-echo segments via,

$${\hat{∝}}_{ij} ( f ) = {\int }_{{x}_{i}}^{{x}_{j}} ∝ ( {x}^{′} , f ) d {x}^{′} + \frac{1}{2} ln [ \frac{T ( f , {x}_{i} )}{T ( f , {x}_{j} )} ] \qquad ( 24 )$$

The second term in the right hand side of equation (24) corresponds to unwanted noise and indicates the contribution from the local structure. As discussed above, a plurality of realizations of RF A-line signals from the same tissue region may be used in employing such a method. The “structure” component is from the contrast agent. As the contrast agent is moving, different realizations of the structure are obtained at different times, allowing various embodiments to take an expectation and remove the distortion due to the structure component. Thus, the expectation of the pulse amplitude spectrum can be formed which effectively averages away this contribution. The frequency dependent form of the attenuation may then be determined without influence of the structure term. This allows a more complex form of attenuation dependence to be modeled (than, for example, provided by Equation (18)). The complexity, however, comes at a cost of greater computational time and space complexities.

It should be noted that the use of contrast agents in some embodiments results in scattering characteristics of the agent that are the same or substantially similar for all locations. If the tissue structure is changed, then the backscatter non-stationarity could be biased from changes in the local tissue scattering characteristics. Using contrast agents thus may provide a ‘reference standard’ type scattering structure and avoid a potential source of bias in the estimate of \hat{x}ᵢⱼ (f). For example, reference scatterers provided in the blood (e.g., by introduction of a gas-based contrast agent) that may act as calibration scattering targets. A microbubble contrast agent signal can be separated from the tissue signal due to a difference in non-linear response. Further, use of contrast agents may be used in connection with fundamental and/or harmonic signals.

With continued reference to FIG. 3, at 312 a parameter is determined. In various embodiments, one or more parameters, for example one or more parameters describing attenuations characteristics of tissue being scanned, may be determined. The parameter may be selected, for example, based on the ability to use the parameter to identify a tissue type. For example, a parameter, such as β, may be determined for one or more A-lines using an ensemble waveform, as discussed above.

At 314 a tissue type is identified using parametric information. A parameter determined along a given A-line may be used to identify tissue represented along the given A-line. For example, based on a known correlation between a value or a range of values for a parameter, A-lines or portions of A-lines having that value or a value that fits within the range may be identified as the particular type of tissue.

At 316, an image is reconstructed. For example, using the identified tissue type or types from 314, an image may be reconstructed for a given A-line. Similarly, images may be reconstructed for a plurality of A-lines, with the individually reconstructed images than combined to provide a reconstructed planar image. In some embodiments, more than 100 A-lines may be used to provide a planar image.

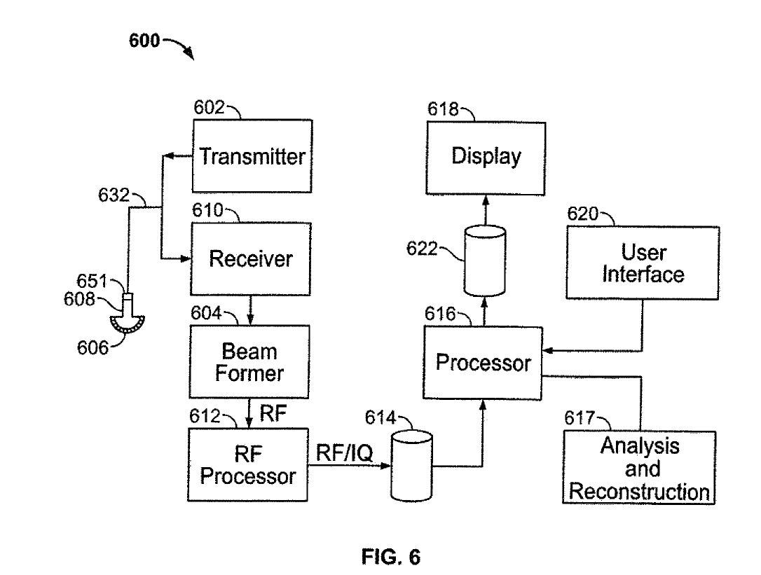

Various embodiments described herein may be implemented in an ultrasound system such as the ultrasound system 600 as shown in FIG. 6. FIG. 6 is a block diagram of an exemplary ultrasound imaging system 600 that is constructed in accordance with various embodiments. The ultrasound system 600 is capable of electrical or mechanical steering of a soundbeam (such as in 3D space) and is configurable to acquire information (e.g., image slices) corresponding to a plurality of 2D representations or images of a region of interest (ROI) in a subject or patient, which may be defined or adjusted as described in more detail herein. The ultrasound system 600 is configurable to acquire 2D images in one or more planes of orientation. The ultrasound system 600 may be embodied in a small-sized system, such as laptop computer, a portable imaging system, a pocket sized system as well as in a larger console-type system.

The ultrasound system 600 includes a transmitter 602 that, under the guidance of a beamformer 604, drives an array of elements 606 (e.g., piezoelectric elements) within a probe 608 to emit pulsed ultrasonic signals, i.e. sound waves, into a body. A variety of geometries may be used. As shown in FIG. 6, the probe 608 may be coupled to the transmitter 602 via the system cable 632 and the connector 651. The sound waves are back-scattered from structures in the body, like blood cells flowing through a blood vessel, to produce echoes that return to the elements 606. The echoes are received by a receiver 610. The received echoes are passed through the beamformer 604, which performs receive beamforming and outputs an RF signal. The RF signal then passes through an RF processor 612. Optionally, the RF processor 612 may include a complex demodulator (not shown) that demodulates the RF signal to form IQ data pairs representative of the echo signals. The RF or IQ signal data may then be routed directly to a buffer 614 for storage.

In the above-described embodiment, the beamformer 604 operates as a transmit and receive beamformer. Optionally, the probe 608 includes a 2D array with sub-aperture receive beamforming inside the probe 608. The beamformer 604 may delay, apodize and/or sum each electrical signal with other electrical signals received from the probe 608. The summed signals represent echoes from the ultrasound beams or lines. The summed signals are output from the beamformer 604 to the RF processor 612. The RF processor 612 may generate different data types, e.g. B-mode, color Doppler (velocity/power/variance), tissue Doppler (velocity), and Doppler energy, for multiple scan planes or different scanning patterns. The RF processor 612 gathers the information (e.g. I/Q, B-mode, color Doppler, tissue Doppler, and Doppler energy information) related to multiple data slices and stores the data information, which may include time stamp and orientation/rotation information, in the buffer 614.

The ultrasound system 600 also includes a processor 616 to process the acquired ultrasound information (e.g., RF signal data or IQ data pairs) and prepare frames of ultrasound information for display on a display 618. For example, the processor 616 may include or have associated therewith an analysis and reconstruction module 617. The analysis and reconstruction module 617, for example, may include an analysis module (e.g., analysis module 220) and a reconstruction module (e.g., reconstruction module 230). The processor 616 is adapted to perform one or more processing operations according to a plurality of selectable ultrasound modalities on the acquired ultrasound data. Acquired ultrasound data may be processed and displayed in real-time during a scanning session as the echo signals are received. Additionally or alternatively, the ultrasound data may be stored temporarily in the buffer 614 during a scanning session and then processed and displayed in an off-line operation.

The processor 616 is connected to a user interface 620 that may control operation of the processor 616 as explained below in more detail. The display 618 may include one or more monitors that present patient information, including diagnostic ultrasound images to the user for diagnosis and analysis. The buffer 614 and/or a memory 622 may store two-dimensional (2D) or three-dimensional (3D) data sets of the ultrasound data, where such 2D and 3D data sets are accessed to present 2D (and/or 3D images). The images may be modified and the display settings of the display 618 may also be manually adjusted using the user interface 620.

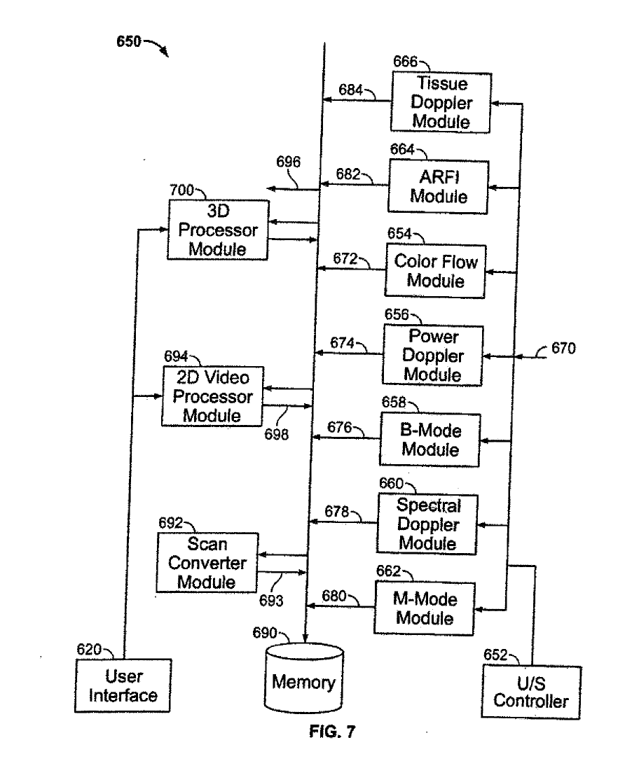

The various components of the ultrasound system 600 may have different configurations. For example, FIG. 7 illustrates an exemplary block diagram of an ultrasound processor module 650, which may be embodied as a portion of the processor 616 shown in FIG. 6. The ultrasound processor module 650 is illustrated conceptually as a collection of sub-modules, but may be implemented utilizing any combination of dedicated hardware boards, DSPs, processors, etc. Alternatively, the sub-modules of FIG. 7 may be implemented utilizing an off-the-shelf PC with a single processor or multiple processors, with the functional operations distributed between the processors. As a further option, the sub-modules of FIG. 7 may be implemented utilizing a hybrid configuration in which certain modular functions are performed utilizing dedicated hardware, while the remaining modular functions are performed utilizing an off-the shelf PC and the like. The sub-modules also may be implemented as software modules within a processing unit.

The operations of the sub-modules illustrated in FIG. 7 may be controlled by a local ultrasound controller 652 or by the processor 616. The sub-modules 654-666 perform mid-processor operations. The ultrasound processor module 650 may receive ultrasound data 670 in one of several forms. In the embodiment of FIG. 7, the received ultrasound data 670 constitutes I,Q data pairs representing the real and imaginary components associated with each data sample. The I,Q data pairs are provided to one or more of a color-flow sub-module 654, a power Doppler sub-module 656, a B-mode sub-module 658, a spectral Doppler sub-module 660 and an M-mode sub-module 662. Optionally, other sub-modules may be included such as an Acoustic Radiation Force Impulse (ARFI) sub-module 664 and a Tissue Doppler (TDE) sub-module 666, among others.

Each of sub-modules 654-666 are configured to process the I,Q data pairs in a corresponding manner to generate color-flow data 672, power Doppler data 674, B-mode data 676, spectral Doppler data 678, M-mode data 680, ARFI data 682, and tissue Doppler data 684, all of which may be stored in a memory 690 (or memory 614 or memory 622 shown in FIG. 6) temporarily before subsequent processing. For example, the B-mode sub-module 658 may generate B-mode data 676 including a plurality of B-mode image planes.

The data 672-684 may be stored, for example, as sets of vector data values, where each set defines an individual ultrasound image frame. The vector data values are generally organized based on the polar coordinate system.

A scan converter sub-module 692 accesses and obtains from the memory 690 the vector data values associated with an image frame and converts the set of vector data values to Cartesian coordinates to generate an ultrasound image frame 693 formatted for display. The ultrasound image frames 693 generated by the scan converter module 692 may be provided back to the memory 690 for subsequent processing or may be provided to the memory 614 or 622.

Once the scan converter sub-module 692 generates the ultrasound image frames 693 associated with, for example, B-mode image data, and the like, the image frames 693 may be restored in the memory 690 or communicated over a bus 696 to a database (not shown), the memory 614, and the memory 622 and/or to other processors.

The scan converted data may be converted into an X,Y format for video display to produce ultrasound image frames. The scan converted ultrasound image frames are provided to a display controller (not shown) that may include a video processor that maps the video to a grey-scale mapping for video display. The grey-scale map may represent a transfer function of the raw image data to displayed grey levels. Once the video data is mapped to the grey-scale values, the display controller controls the display 618 (shown in FIG. 6), which may include one or more monitors or windows of the display, to display the image frame. The image displayed in the display 618 is produced from image frames of data in which each datum indicates the intensity or brightness of a respective pixel in the display.

Referring again to FIG. 7, a 2D video processor sub-module 694 combines one or more of the frames generated from the different types of ultrasound information. For example, the 2D video processor sub-module 694 may combine a different image frames by mapping one type of data to a grey map and mapping the other type of data to a color map for video display. In the final displayed image, color pixel data may be superimposed on the grey scale pixel data to form a single multi-mode image frame 698 (e.g., functional image) that is again re-stored in the memory 690 or communicated over the bus 696. Successive frames of images may be stored as a cine loop, for example in the memory 690. The cine loop represents a first in, first out circular image buffer to capture image data that is displayed to the user. The user may freeze the cine loop by entering a freeze command at the user interface 620. The user interface 620 may include, for example, a keyboard and mouse and all other input controls associated with inputting information into the ultrasound system 600 (shown in FIG. 6).

A 3D processor sub-module 700 is also controlled by the user interface 620 and accesses the memory 690 to obtain 3D ultrasound image data and to generate three dimensional images, such as through volume rendering or surface rendering algorithms as are known. The three dimensional images may be generated utilizing various imaging techniques, such as ray-casting, maximum intensity pixel projection and the like.

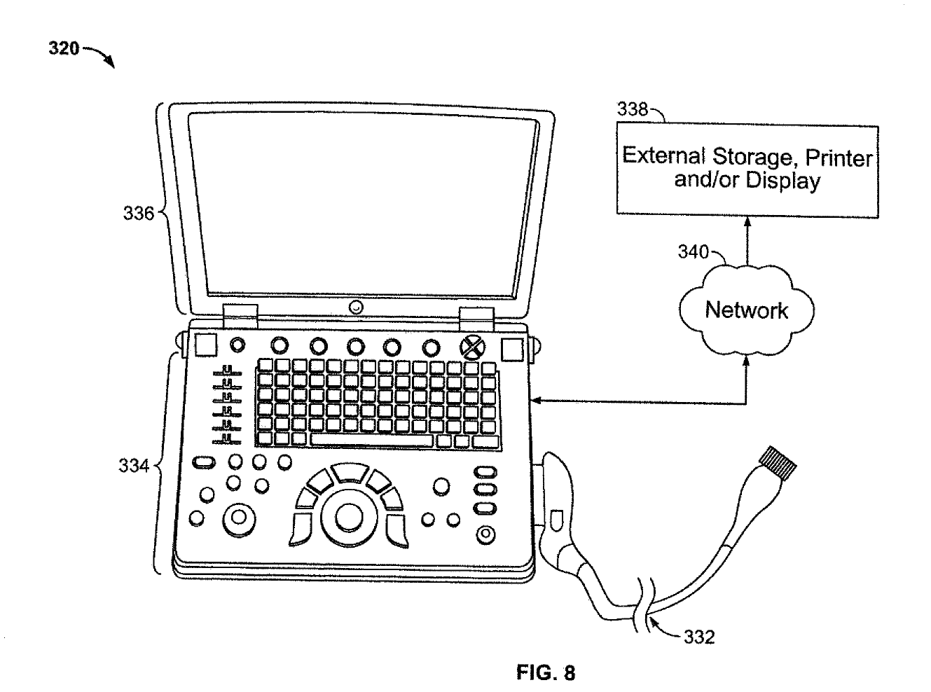

FIG. 8 illustrates a 3D-capable miniaturized ultrasound system 320 having an ultrasound transducer 332 that may be configured to acquire 3D ultrasonic data or multi-plane ultrasonic data. For example, the ultrasound transducer 332 may have an array of acoustic elements. A user interface 334 (that may also include an integrated display 336) is provided to receive commands from an operator. As used herein, “miniaturized” means that the ultrasound system 320 is a handheld or hand-carried device or is configured to be carried in a person's hand, pocket, briefcase-sized case, or backpack. For example, the ultrasound system 320 may be a hand-carried device having a size of a typical laptop computer. The ultrasound system 320 is easily portable by the operator. The integrated display 336 (e.g., an internal display) is configured to display, for example, one or more medical images.

The ultrasonic data may be sent to an external device 338 via a wired or wireless network 340 (or direct connection, for example, via a serial or parallel cable or USB port). In some embodiments, the external device 338 may be a computer or a workstation having a display, or the DVR of the various embodiments. Alternatively, the external device 338 may be a separate external display or a printer capable of receiving image data from the hand carried ultrasound system 320 and of displaying or printing images that may have greater resolution than the integrated display 336.

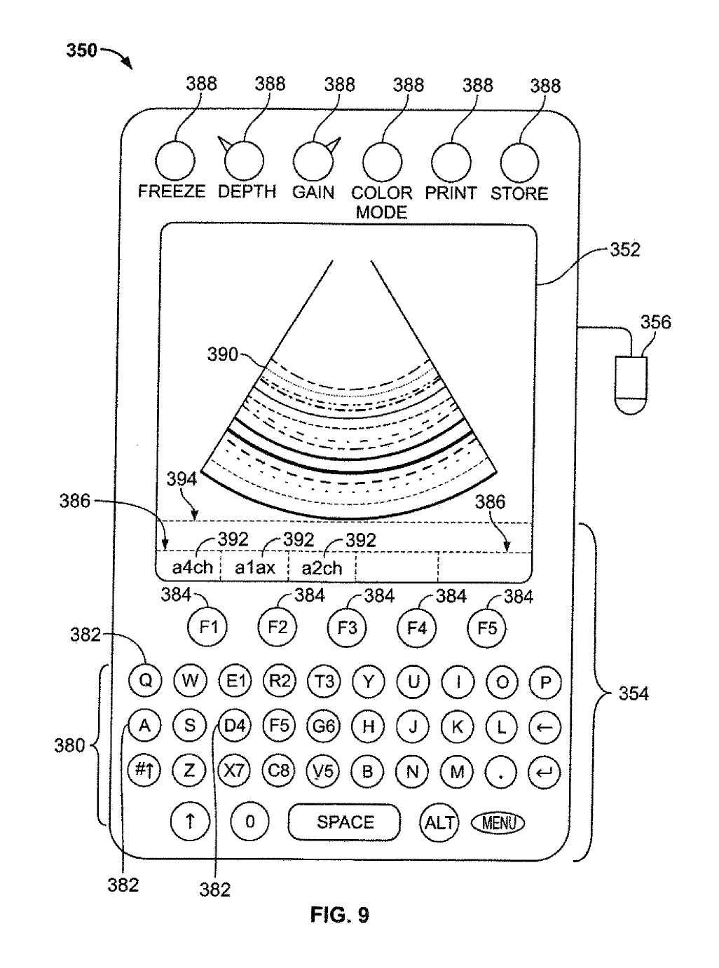

FIG. 9 illustrates a hand carried or pocket-sized ultrasound imaging system 350 wherein the display 352 and user interface 354 form a single unit. By way of example, the pocket-sized ultrasound imaging system 350 may be a pocket-sized or hand-sized ultrasound system approximately 2 inches wide, approximately 4 inches in length, and approximately 0.5 inches in depth and weighs less than 3 ounces. The pocket-sized ultrasound imaging system 350 generally includes the display 352, user interface 354, which may or may not include a keyboard-type interface and an input/output (I/O) port for connection to a scanning device, for example, and an ultrasound transducer 356. The display 352 may be, for example, a 320×320 pixel color LCD display (on which a medical image 390 may be displayed). A typewriter-like keyboard 380 of buttons 382 may optionally be included in the user interface 354.

Multi-function controls 384 may each be assigned functions in accordance with the mode of system operation (e.g., displaying different views). Therefore, each of the multi-function controls 384 may be configured to provide a plurality of different actions. Label display areas 386 associated with the multi-function controls 384 may be included as necessary on the display 352. The system 350 may also have additional keys and/or controls 388 for special purpose functions, which may include, but are not limited to “freeze,” “depth control,” “gain control,” “color-mode,” “print,” and “store.”

One or more of the label display areas 386 may include labels 392 to indicate the view being displayed or allow a user to select a different view of the imaged object to display. The selection of different views also may be provided through the associated multi-function control 384. The display 352 may also have a textual display area 394 for displaying information relating to the displayed image view (e.g., a label associated with the displayed image).

It should be noted that the various embodiments may be implemented in connection with miniaturized or small-sized ultrasound systems having different dimensions, weights, and power consumption.

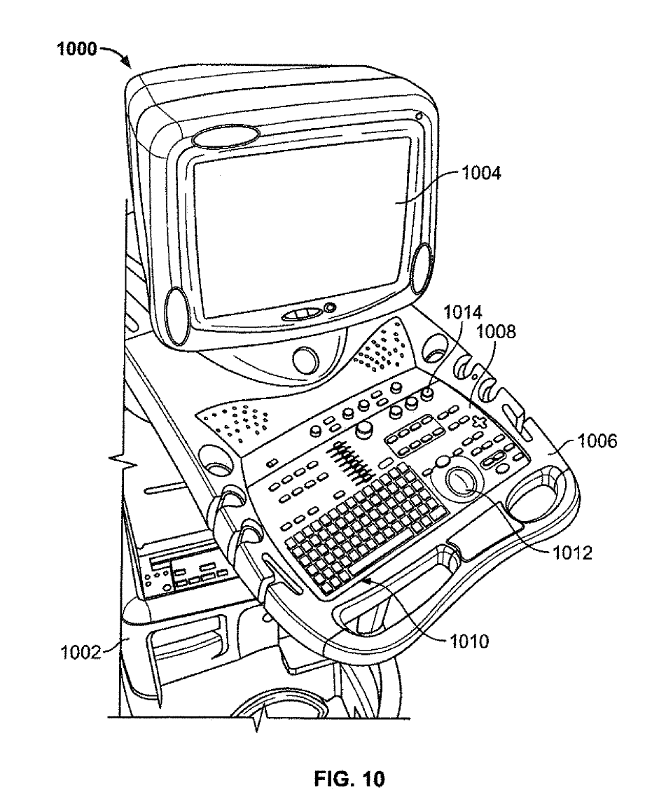

FIG. 10 illustrates an ultrasound imaging system 1000 provided on a movable base 1002. The portable ultrasound imaging system 1000 may also be referred to as a cart-based system. A display 1004 and user interface 1006 are provided and it should be understood that the display 1004 may be separate or separable from the user interface 1006. The user interface 1006 may optionally be a touchscreen, allowing the operator to select options by touching displayed graphics, icons, and/or the like.

The user interface 1006 also includes control buttons 1008 that may be used to control the portable ultrasound imaging system 1000 as desired or needed, and/or as typically provided. The user interface 1006 provides multiple interface options that the user may physically manipulate to interact with ultrasound data and other data that may be displayed, as well as to input information and set and change scanning parameters and viewing angles, etc. For example, a keyboard 1010, trackball 1012 and/or multi-function controls 1014 may be provided.

Thus, various embodiments provide for improved imaging. For example, improved identification of tissue types may be achieved by identifying parameters that are not sufficiently identified by conventional ultrasound systems. For example, some embodiments may provide improved identification of fat content, fibrous content, and/or tumors of liver tissue.

The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a floppy disk drive, optical disk drive, and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), ASICs, logic circuits, and any other circuit or processor capable of executing the functions described herein. The above examples are exemplary only, and are thus not intended to limit in any way the definition and/or meaning of the term “computer.”

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the invention. For example, a module or system may include a computer processor, controller, or other logic-based device that performs operations based on instructions stored on a tangible and non-transitory computer readable storage medium, such as a computer memory. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments of the invention without departing from their scope. While the dimensions and types of materials described herein are intended to define the parameters of the various embodiments of the invention, the embodiments are by no means limiting and are exemplary embodiments. Many other embodiments will be apparent to those of skill in the art upon reviewing the above description. The scope of the various embodiments of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the various embodiments of the invention, and also to enable any person skilled in the art to practice the various embodiments of the invention, including making and using any devices or systems and performing any incorporated methods. The patentable scope of the various embodiments of the invention is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

What is claimed is:

1. A method for forming an ultrasound image, the method comprising:
obtaining ultrasound information, the ultrasound information including anatomy information representative of a portion of anatomy to be imaged and artifact information representative of a coherent interference artifact, wherein a contrast agent has been associated with the coherent interference artifact, wherein obtaining the ultrasound information comprises taking a plurality of readings along a single line, whereby, due to association with the contrast agent, the coherent interference artifact appears in different realizations in the plurality of readings;
suppressing the artifact information representative of the coherent interference artifact, using the plurality of readings along the single line, to form revised ultrasound information; and
reconstructing the ultrasound image using the revised ultrasound information.

2. A method in accordance with claim 1, wherein the contrast agent is associated with the coherent interference artifact but is not substantially associated with the portion of anatomy to be imaged.

3. A method in accordance with claim 1 further comprising determining a parameter from the revised ultrasound information, wherein reconstructing the image comprises reconstructing a parametric image using the parameter.

4. A method in accordance with claim 3, wherein the parameter is based upon a frequency dependent attenuation characteristic, the attenuation characteristic corresponding to the attenuation of an ultrasound wave in tissue.

5. A method in accordance with claim 4, wherein a frequency is determined using the relationship:
$${\overline{f}}_{τ} = \frac{{\int }_{0}^{∞} f {\vert {S}_{τ} ( f ) \vert}^{2} d f}{{\int }_{0}^{∞} {\vert {S}_{τ} ( f ) \vert}^{2} d f} = \frac{1}{2 π} \frac{{\int }_{τ - \frac{L}{2}}^{τ + \frac{L}{2}} Φ ( t ) {a}^{2} ( t ) d t}{{\int }_{τ - \frac{L}{2}}^{τ + \frac{L}{2}} {a}^{2} ( t ) d t} ,$$
where L is a length of a radio frequency (RF) line segment in the ultrasound information centered at τ.

6. A method in accordance with claim 4, wherein determining the parameter includes determining β, where β is defined by the relationship α(f)≈βf, where α corresponds to frequency-dependent amplitude attenuation, and f describes a frequency.

7. A method in accordance with claim 1, wherein suppressing the artifact information to form the revised ultrasound information includes combining a plurality of differences of non-sequential readings from the plurality of readings along the single line.

8. A tangible and non-transitory computer readable medium comprising one or more computer software modules configured to direct a processor to:
receive ultrasound information from an ultrasound detector, the ultrasound information including anatomy information representative of a portion of anatomy to be imaged and artifact information representative of a coherent interference artifact, wherein a contrast agent has been associated with the coherent interference artifact, wherein the ultrasound information comprises information obtained from a plurality of readings along a single line, whereby, due to association with the contrast agent, the coherent interference artifact appears in different realizations in the plurality of readings;
suppress the artifact information representative of the coherent interference artifact to form revised ultrasound information, wherein the artifact information is suppressed by use of the plurality of readings along the single line; and
reconstruct the ultrasound image using the revised ultrasound information.

9. The tangible and non-transitory computer readable medium of claim 8, wherein the contrast agent is associated with the coherent interference artifact but is not substantially associated with the portion of anatomy to be imaged.

10. The tangible and non-transitory computer readable medium of claim 8, wherein the one or more software modules are further configured to direct the processor to determine a parameter from the revised ultrasound information and to reconstruct a parametric image using the parameter.

11. The tangible and non-transitory computer readable medium of claim 10, wherein the parameter is based upon a frequency dependent attenuation characteristic, the attenuation characteristic corresponding to the attenuation of an ultrasound wave in tissue.

12. The tangible and non-transitory computer readable medium of claim 11, wherein the one or more software modules are further configured to direct the processor to determine a frequency using the relationship
$${\overline{f}}_{τ} = \frac{{\int }_{0}^{∞} f {\vert {S}_{T} ( f ) \vert}^{2} d f}{{\int }_{0}^{∞} {\vert {S}_{T} ( f ) \vert}^{2} d f} = \frac{1}{2 π} \frac{{\int }_{τ - \frac{L}{2}}^{τ + \frac{L}{2}} Φ ( t ) {a}^{2} ( t ) d t}{{\int }_{τ - \frac{L}{2}}^{τ + \frac{L}{2}} {a}^{2} ( t ) d t} ,$$
where L is a length of a radio frequency (rf) line segment in the ultrasound information centered at τ.

13. The tangible and non-transitory computer readable medium of claim 11, wherein the one or more software modules are further configured to direct the processor to determine β, where β is defined by the relationship α(f)≈βf, where α corresponds to frequency-dependent amplitude attenuation, and f describes a frequency.

14. The tangible and non-transitory computer readable medium of claim 8, wherein the one or more software modules are further configured to direct the processor to combine a plurality of differences of non-sequential readings from the plurality of readings along the single line to suppress the artifact information representative of the coherent interference artifact.

15. A system comprising:
an acquisition module comprising an ultrasound probe, the acquisition module configured to acquire ultrasound information including anatomy information representative of a portion of anatomy to be imaged and artifact information representative of a coherent interference artifact, wherein a contrast agent has been associated with the coherent interference artifact, wherein the ultrasound information comprises information obtained by a plurality of readings along a single line, whereby, due to association with the contrast agent, the coherent interference artifact appears in different realizations in the plurality of readings;
an analysis module including a processing unit, the analysis module configured to receive the ultrasound information from the acquisition module and to suppress the artifact information representative of the coherent interference artifact to provide revised ultrasound information, wherein the analysis module is configured to suppress the artifact information by use of the plurality of readings along the single line; and
a reconstruction module including a processing unit, the reconstruction module configured to reconstruct an image using the revised ultrasound information from the analysis module.

16. A system in accordance with claim 15, wherein the contrast agent is associated with the coherent interference artifact but is not substantially associated with the portion of anatomy to be imaged.

17. A system in accordance with claim 15, wherein the analysis module is configured to determine a parameter from the revised ultrasound information, and wherein the reconstruction module is configured to reconstruct the image using the parameter.

18. A system in accordance with claim 17, wherein the parameter is based upon a frequency dependent attenuation characteristic, the attenuation characteristic corresponding to the attenuation of an ultrasound wave in tissue.

19. A system in accordance with claim 18 wherein the analysis module is configured to determine a frequency using the relationship
$${\overline{f}}_{τ} = \frac{{\int }_{0}^{∞} f {\vert {S}_{τ} ( f ) \vert}^{2} d f}{{\int }_{0}^{∞} {\vert {S}_{τ} ( f ) \vert}^{2} d f} = \frac{1}{2 π} \frac{{\int }_{τ - \frac{L}{2}}^{τ + \frac{L}{2}} Φ ( t ) {a}^{2} ( t ) d t}{{\int }_{τ - \frac{L}{2}}^{τ + \frac{L}{2}} {a}^{2} ( t ) d t} ,$$
where L is a length of a radio frequency (rf) line segment in the ultrasound information centered at τ.

20. A system in accordance with claim 18 wherein the analysis module is configured to determine β, where β is defined by the relationship α(f)≈βf, where α corresponds to frequency-dependent amplitude attenuation, and f describes a frequency.

21. A system in accordance with claim 15, wherein the analysis module is configured to combine a plurality of differences of non-sequential readings from the plurality of readings along the single line to suppress the artifact information from the ultrasound information to provide the revised ultrasound information.
